(19) United States
(12) Patent Application Publication
(10) Pub. No.: US 2018/0169372 A1
(43) Pub. Date: Jun. 21, 2018

(54) SYSTEMS AND METHODS FOR GENERATING PINK NOISE STIMULATION

(71) Applicant: PACESETTER, INC., Sylmar, CA (US)

(72) Inventors: Gene A. Bornzin, Simi Valley, CA (US); Stuart Rosenberg, Castaic, CA (US)

(21) Appl. No.: 15/387,170

(22) Filed: Dec. 21, 2016

(51) Int. Cl. A61M 21/02

(52) U.S. Cl. CPC A61M 21/02; A61M 2021/0027

(57) ABSTRACT

The present disclosure provides systems and methods for delivering pink noise stimulation. A neurostimulation system includes a stimulation lead comprising a plurality of contacts, and a pulse generator communicatively coupled to the stimulation lead and configured to generate white noise, filter the generated white noise to generate pink noise, apply a high pass filter to the pink noise, apply thresholding to the filtered pink noise, convert each excursion in the thresholded pink noise into a square pulse to generate a final pink noise signal, and cause stimulation to be delivered by the stimulation lead based on the final pink noise signal.

### A. FIELD OF THE DISCLOSURE

The present disclosure relates generally to neurostimulation systems, and more particularly to generating a pink noise signal for use in neurostimulation.

### B. BACKGROUND ART

Neurostimulation is an established neuromodulation therapy for the treatment of movement disorders. For example, neurostimulation has been shown to improve cardinal motor symptoms of Parkinson's Disease (PD), such as bradykinesia, rigidity, and tremors. Types of neurostimulation include deep brain stimulation (DBS) and spinal cord stimulation (SCS).

Pink noise (i.e., noise having a power spectral density inversely proportional to frequency) has been shown to have advantageous effects in neurostimulation. However, generating pink noise for neurostimulation is challenging. Specifically, pink noise is generally a continuous function. However, pulse generators in neurostimulation systems typically provide tonic stimuli and/or burst stimuli of fixed programmable frequency and pulse width.

### BRIEF SUMMARY OF THE DISCLOSURE

In one embodiment, the present disclosure is directed to a neurostimulation system for delivering pink noise stimulation. The neurostimulation system includes a stimulation lead comprising a plurality of contacts, and a pulse generator communicatively coupled to the stimulation lead and configured to generate white noise, filter the generated white noise to generate pink noise, apply a high pass filter to the pink noise, apply thresholding to the filtered pink noise, convert each excursion in the thresholded pink noise into a square pulse to generate a final pink noise signal, and cause stimulation to be delivered by the stimulation lead based on the final pink noise signal.

In another embodiment, the present disclosure is directed to a computer-implemented method for generating a pink noise signal. The method includes generating, using a computing device, white noise using a random number generator, filtering, using the computing device, the generated white noise to generate pink noise, applying, using the computing device, a high pass filter to the pink noise, applying, using the computing device, thresholding to the filtered pink noise, and converting, using the computing device, each excursion in the thresholded pink noise into a square pulse to generate a final pink noise signal.

In another embodiment, the present disclosure is directed to a computer-implemented method for generating a pink noise signal. The method includes determining, using a computing device, a frequency band, determining, using the computing device, a number of nodes and a spacing scheme for the nodes, computing, using the computing device, a location of each node within the frequency band, computing, using the computing device, a weight for each node, initiating, using the computing device, a startup signal, computing, after a predetermined period of time, a nominal amplitude of a current stimulus pulse as a sum-product of amplitudes of previous pulses by the computed weights, selecting a pseudorandom number, and computing a final stimulus amplitude as a sum of the nominal amplitude and the pseudorandom number to generate the pink noise signal.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIG. 2 is a block diagram of one embodiment of a computing device that may be used to generate pink noise.

FIG. 3 is a flow chart of one embodiment of a method for generating a pink noise signal.

FIG. 4 is a graph showing an example of generated white noise.

FIG. 5 is a graph showing an example of white noise filtered using a 1/f^{α} filter.

FIG. 6 is a graph showing an example of pink noise passed through a high pass filter.

FIG. 7 is a graph showing the frequency content of the high pass filtered pink noise of the graph shown in FIG. 6.

FIG. 8 is a graph showing thresholding applied to fifteen milliseconds (ms) of the high pass filtered pink noise from the graph shown in FIG. 6.

FIG. 9 is a graph showing the generation of square pulses.

FIG. 10 is a graph showing the frequency content of the final pink noise signal of the graph shown in FIG. 9.

FIG. 11 is a graph showing a modified final pink noise signal.

FIG. 12 is a graph showing the frequency content of the modified final pink noise signal of the graph shown in FIG. 11.

FIG. 13 is a graph showing another modified final pink noise signal.

FIG. 14 is a graph showing the frequency content of the modified final pink noise signal of the graph shown in FIG. 13.

FIG. 15 is a graph showing another modified final pink noise signal.

FIG. 16 is a graph showing the frequency content of the modified final pink noise signal of the graph shown in FIG. 15.

FIG. 17 is a graph showing another modified final pink noise signal.

FIG. 18 is a graph showing the frequency content of the modified final pink noise signal of the graph shown in FIG. 17.

FIG. 19 is a graph showing another modified final pink noise signal.

FIG. 20 is a graph showing the frequency content of the modified final pink noise signal of the graph shown in FIG. 19.

FIG. 21 is a graph showing another modified final pink noise signal.

FIG. 22 is a graph showing the frequency content of the modified final pink noise signal of the graph shown in FIG. 21.

FIGS. 23A and 23B are a flow chart of another embodiment of a method for generating a pink noise signal.

FIG. 24 is a first graph showing node locations, a second graph showing node weights, and a third graph showing a node history map.

FIG. 25 is a graph showing time-series amplitudes.

FIG. 26 is a first graph showing a FFT of time-series amplitudes shown in FIG. 25 and a second graph showing a FFT of a pulse train generated based on those amplitudes.

Corresponding reference characters indicate corresponding parts throughout the several views of the drawings.

### DETAILED DESCRIPTION OF THE DISCLOSURE

The present disclosure provides systems and methods for generating a pink noise signal for use in a neurostimulation system. The pink noise signal includes a plurality of pulses that approximate true pink noise. The pink noise signal may be generated, for example, using an internal pulse generator or an external pulse generator.

Neurostimulation systems are devices that generate electrical pulses and deliver the pulses to nerve tissue of a patient to treat a variety of disorders. One category of neurostimulation systems is deep brain stimulation (DBS). In DBS, electrical pulses are delivered to parts of a subject's brain, for example, for the treatment of movement and effective disorders such as PD and essential tremor. Another category of neurostimulation systems is spinal cord stimulation (SCS).

Neurostimulation systems generally include a pulse generator and one or more leads. A stimulation lead includes a lead body of insulative material that encloses wire conductors. The distal end of the stimulation lead includes multiple electrodes, or contacts, that are electrically coupled to the wire conductors. The proximal end of the lead body includes multiple terminals (also electrically coupled to the wire conductors) that are adapted to receive electrical pulses. In DBS systems, the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted within a subcutaneous pocket created during the implantation procedure.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

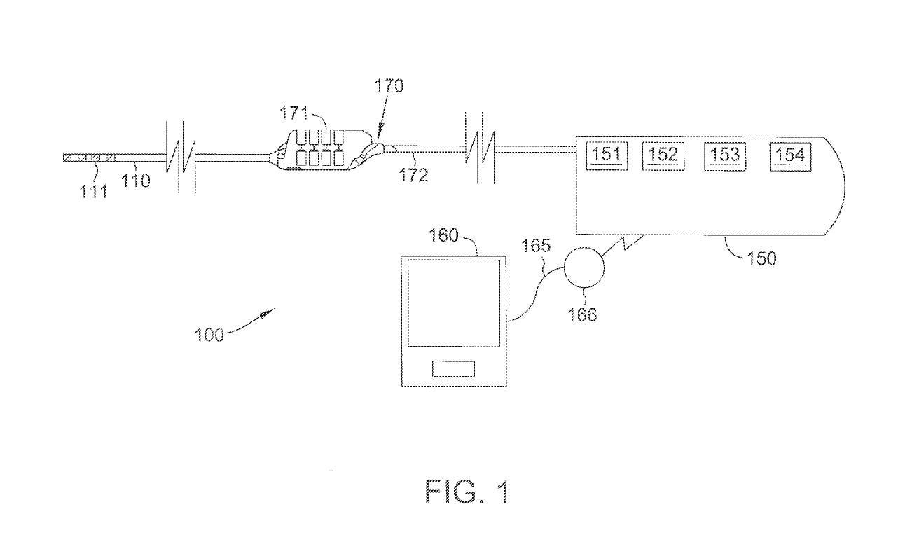

Referring now to the drawings, and in particular to FIG. 1, a stimulation system is indicated generally at 100. Stimulation system 100 generates electrical pulses for application to tissue of a patient, or subject, according to one embodiment. System 100 includes an implantable pulse generator (IPG) 150 that is adapted to generate electrical pulses for application to tissue of a patient. Alternatively, system 100 may include an external pulse generator (EPG) positioned outside the patient's body. IPG 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from IPG 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within IPG 150. Different pulses on different electrodes may be generated using a single set of pulse generating circuitry using consecutively generated pulses according to a “multi-stimset program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pulse generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also, sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

Controller device 160 may be implemented to recharge battery 153 of IPG 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller device 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of IPG 150. The charging circuitry may also communicate status messages to controller device 160 during charging operations using pulse-loading or any other suitable technique. For example, controller device 160 may communicate the coupling status, charging status, charge completion status, etc.

External controller device 160 is also a device that permits the operations of IPG 150 to be controlled by user after IPG 150 is implanted within a patient, although in alternative embodiments separate devices are employed for charging and programming. Also, multiple controller devices may be provided for different types of users (e.g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stimset during execution of program), etc. In the methods and systems described herein, parameters may include, for example, a number of pulses in a burst (e.g., 3, 4, or 5 pulses per burst), an intra-burst frequency (e.g., 130 Hz), an inter-burst frequency (e.g., 3-20 Hz), and a delay between a first and second burst (e.g., less than 1 millisecond (ms)).

IPG 150 modifies its internal parameters in response to the control signals from controller device 160 to vary the stimulation characteristics of stimulation pulses transmitted through stimulation lead 110 to the tissue of the patient. Neurostimulation systems, stimsets, and multi-stimset programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference. Example commercially available neurostimulation systems include the EON MINI™ pulse generator and RAPID PROGRAMMER™ device from St. Jude Medical, Inc. (Plano, Tex.).

The systems and methods described herein facilitate generating pink noise stimulation for use in neurostimulation systems. In some embodiments, the pink noise is generated using generated pulses at random intervals, pulse durations, and amplitudes. These stimuli may be stored in a generator (e.g., IPG 150 or an external pulse generator), such that the generator ensures that each pulse is delivered in a specific order, after a specific interval following a previous pulse, with each pulse having a specific amplitude and duration. This provides a periodic pseudo-random stimuli that if looped over a sufficient time interval, appears to be a random stimuli. Alternatively, a real-time contiguous generation of pseudo-random stimuli can be achieved, as described herein.

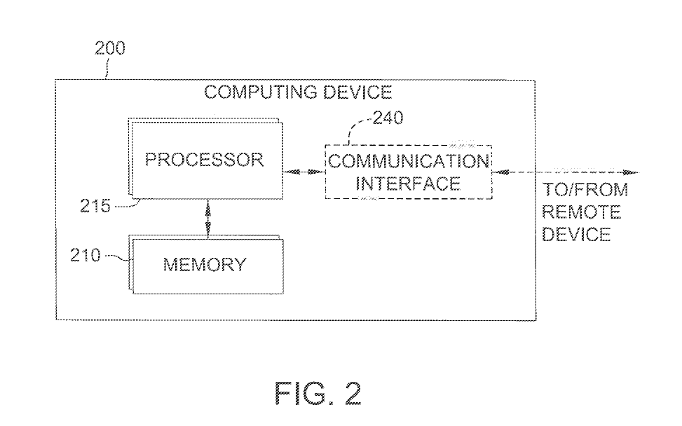

FIG. 2 is a block diagram of one embodiment of a computing device 200 that may be used to generate pink noise as described herein. Computing device 200 may be included, for example, within an IPG (e.g., IPG 150) or an external pulse generator. The systems and methods described herein utilize a random number generator. For example, a random seed may be generated using noise from a diode. Accordingly, in some embodiments, an external pulse generator may be preferable to an IPG.

In this embodiment, computing device 200 includes at least one memory device 210 and a processor 215 that is coupled to memory device 210 for executing instructions. In some embodiments, executable instructions are stored in memory device 210. In the illustrated embodiment, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

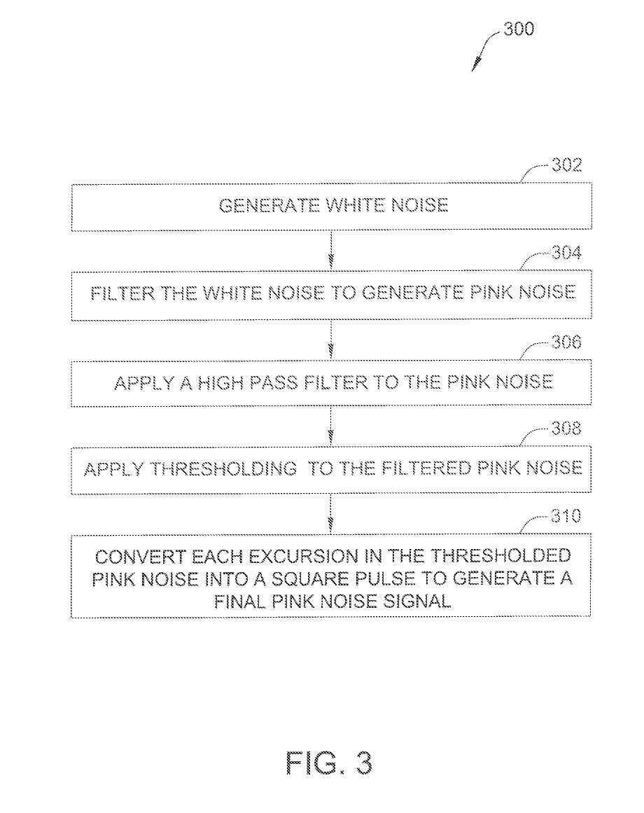

FIG. 3 is a flow chart of one embodiment of a method 300 for generating a pink noise signal. Method 300 may be performed, for example, using computing device 200 (shown in FIG. 2). At block 302, white noise is generated. White noise may be generated, for example, by generating a set of random numbers of equal probability over a predefined range and resolution. For example, white noise may be generated using a predefined range of 0 to 8195 and sampling at 10,000 samples per second.

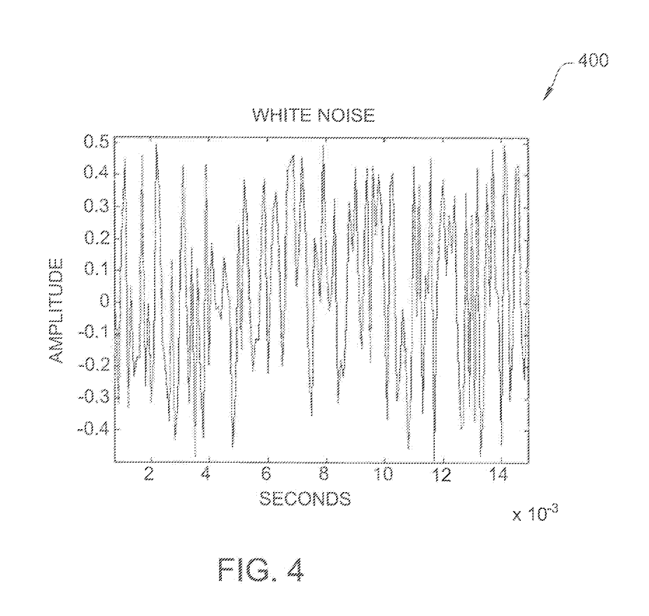

FIG. 4 is a graph 400 showing an example of generated white noise. For graph 400, the noise is sampled at 10,000 samples per second, and each sample is a random number having an equal probability between 0.5 and −0.5.

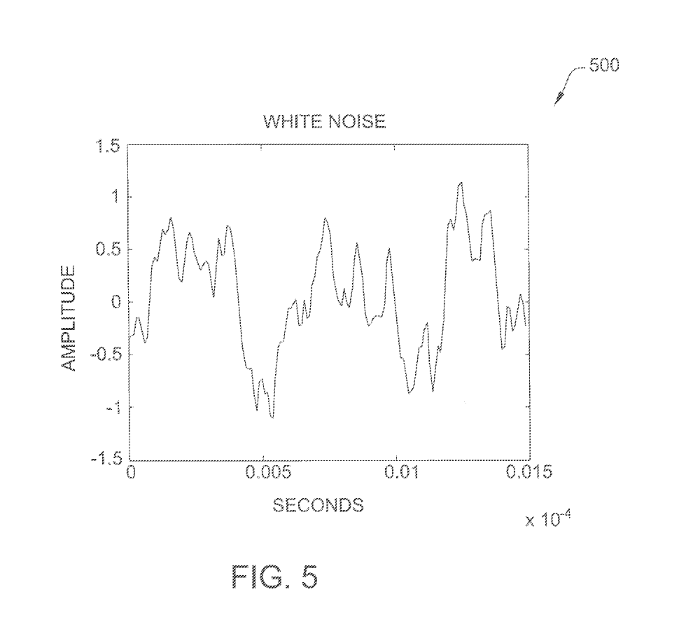

Returning to FIG. 3, at block 304, the white noise generated at block 302 is filtered to create pink noise by weighing the frequency content of the white noise using a 1/f^{α} filter, where f is frequency, and a is a predetermined constant (e.g., 1). The filtering may be accomplished using a fast Fourier transform (FFT) filter or a digital filter. For example, when using a FFT filter, a FFT is applied to the signal, the transformed signal in multiplied by 1/f^{α} symmetrically about the Nyquist frequency (e.g., 5000 Hertz (Hz)) in the frequency domain, and the inverse FFT is applied to return to the time domain. FIG. 5 is a graph 500 showing an example of white noise filtered using a 1/f^{α} filter.

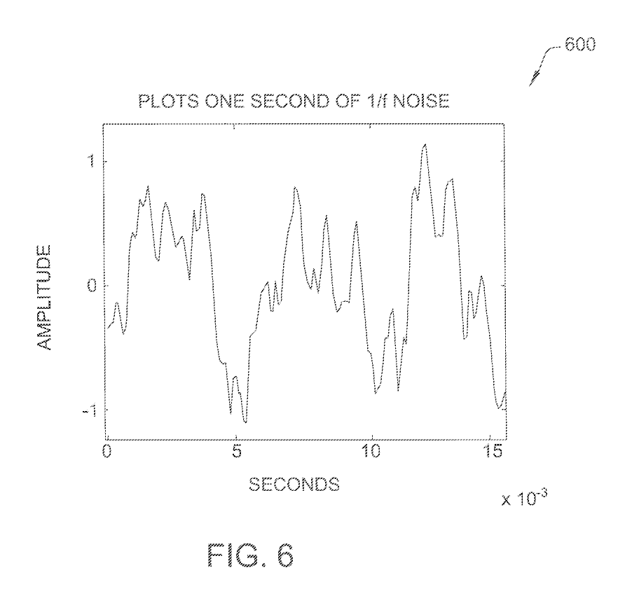

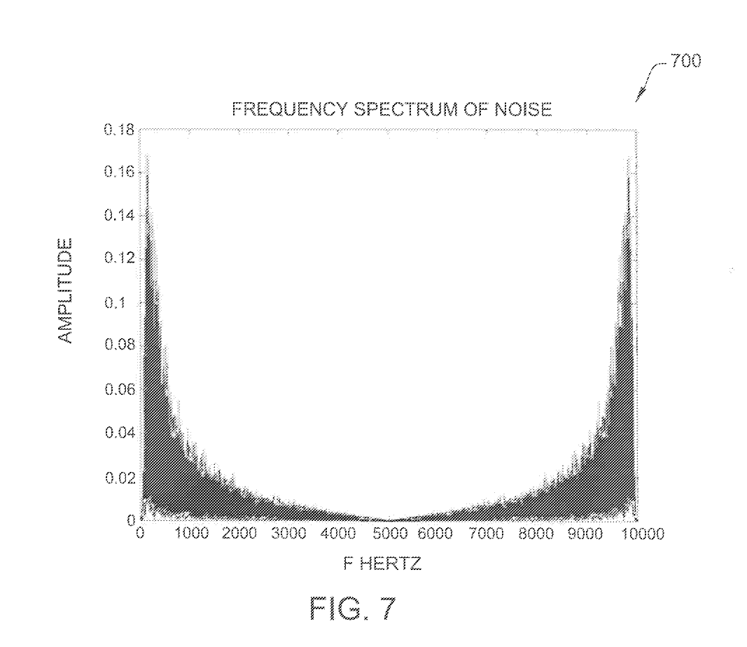

Returning to FIG. 3, at block 306, a high pass filter is applied to the pink noise generated at block 304 to eliminate low frequency content. For example, the pink noise generated at block 304 may be filtered both forwards and backwards using a 120 Hz Butterworth filter. Filtering both forwards and backwards removes phase lags induced by filtering, creates fourth order filtering, and removed DC and low frequency content that may result in excessively long stimuli. Alternatively, any suitable high pass filtering may be used at block 306. FIG. 6 is a graph 600 showing an example of pink noise passed through a high pass filter. FIG. 7 is a graph 700 showing the frequency content of the high pass filtered pink noise of graph 600.

Returning to FIG. 3, at block 308, thresholding is applied to the high pass filtered pink noise generated at block 306. Specifically, thresholding is applied such that only positive and negative values exceeding a predetermined threshold remain, and all other values are replaced with a midpoint of the pink noise. The threshold may be, for example, on the order of ⅛^{th} of the range of the pink noise.

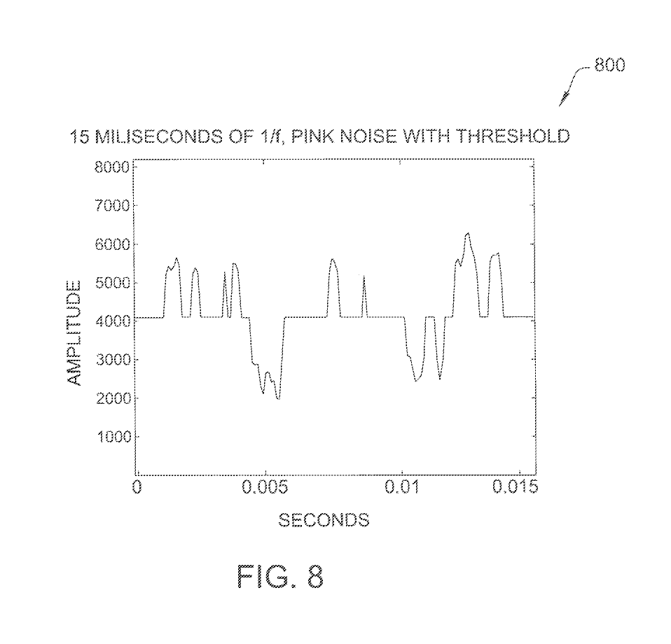

FIG. 8 is a graph 800 showing thresholding applied to fifteen milliseconds (ms) of the high pass filtered pink noise shown in graph 600. The high pass filtered pink noise is scaled between 0 and 8191, with a midpoint of approximately 4096. Then, thresholding is applied such that values between 5096 (i.e., 4096+1000) and 3096 (i.e., 4096-1000) are set to 4096, while any other values are maintained.

Returning to FIG. 3, at block 310, each excursion (i.e., each portion of the signal that deviates from the midpoint value) is converted into a square pulse to generate a final pink noise signal. In this embodiment, for each excursion, the average value for that excursion is calculated, and all samples for that excursion are replaced with a modified value that is equal to the average value. Alternatively, excursions may be converted into square pulses using any suitable algorithm.

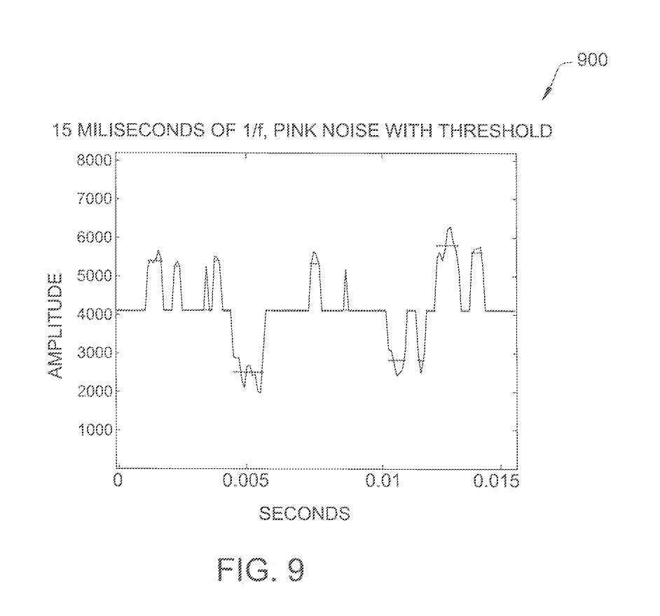

FIG. 9 is a graph 900 showing the generation of square pulses. In graph 900, each asterisk is a modified value equal to the average value for the associated excursion. In this embodiment, single point excursions are ignored. Each set of modified values forms a square pulse that is compatible for use with a stimulation device. Accordingly, a generator (e.g., IPG 150 or an external pulse generator) can control delivery of stimulation based on the square pulses in the final pink noise signal.

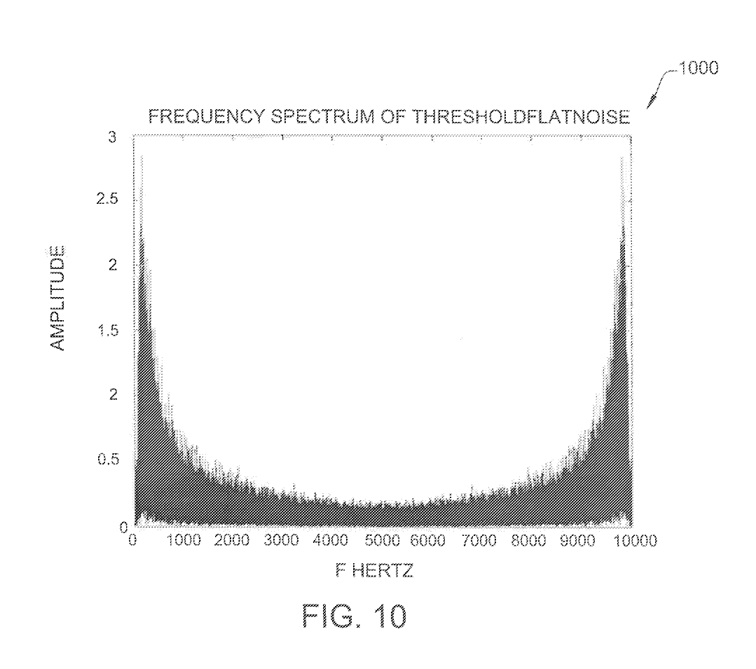

FIG. 10 is a graph 1000 showing the frequency content of the final pink noise signal of graph 900. As compared to graph 700 (shown in FIG. 7), and due to the square pulses, the frequency content in graph 1000 maintains the 1/f characteristics, but also includes high frequency content.

In the final pink noise signal of graph 900, intervals between pulses, pulse amplitudes, and pulse widths are random. That is, the intervals between pulses, the pulse amplitudes, and the pulse widths are not further adjusted or controlled. However, in some embodiments, at least one of the intervals between pulses, the pulse amplitudes, and the pulse widths of the square pulses in the final pink noise signal are controlled to generate a modified final pink noise signal. In such embodiments, the modified final pink noise signal is used by the generator to control delivery of stimulation. Further, controlling intervals between pulses, pulse amplitudes, and/or pulse widths reduces computational complexity for the generation of pink noise, and simplifies the software and hardware needed to generate the pink noise.

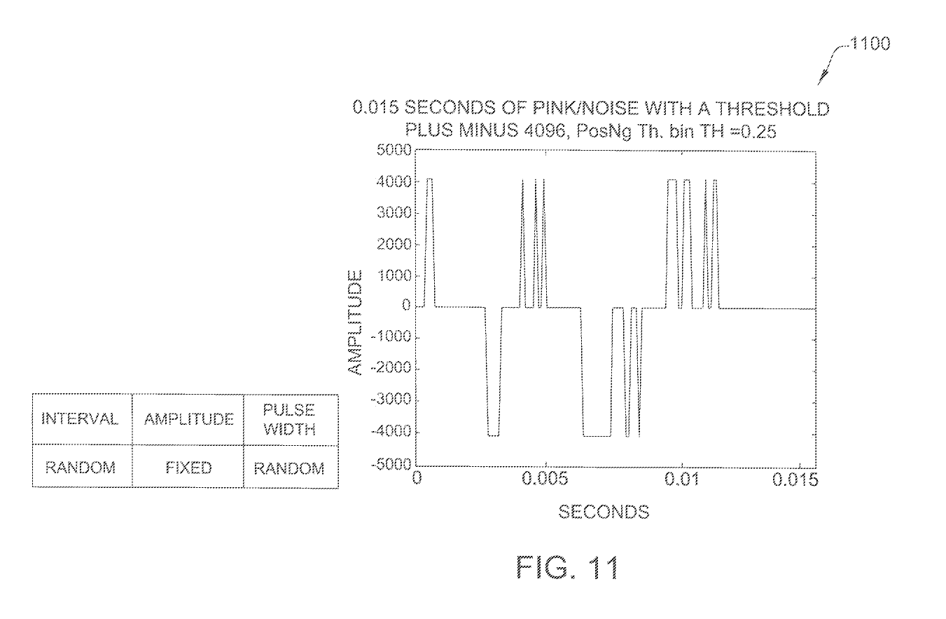

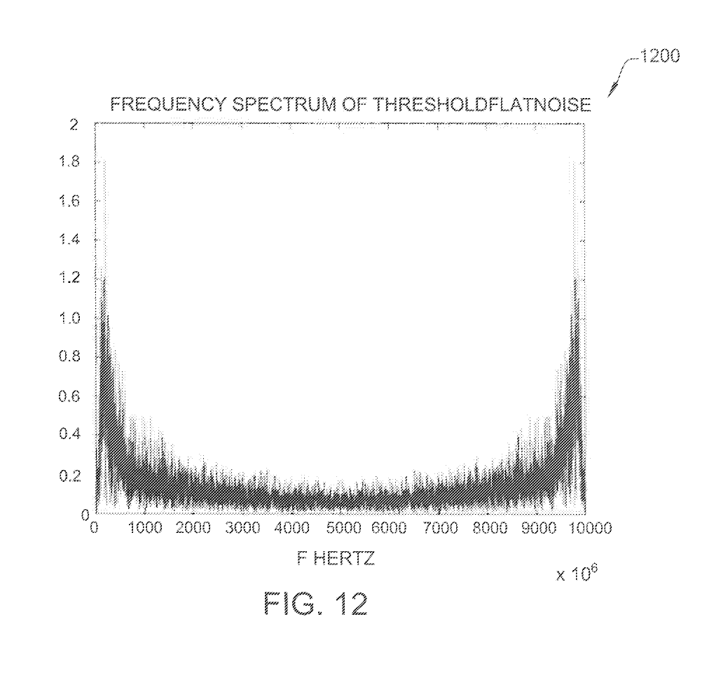

For example, FIG. 11 is a graph 1100 showing a modified final pink noise signal in which the amplitude of each pulse is set equal to a predetermined value (e.g., 4095) while maintaining the polarity of the original pulse. FIG. 12 is a graph 1200 showing the frequency content of the modified final pink noise signal of graph 1100. The frequency content in graph 1200 maintains the 1/f characteristics.

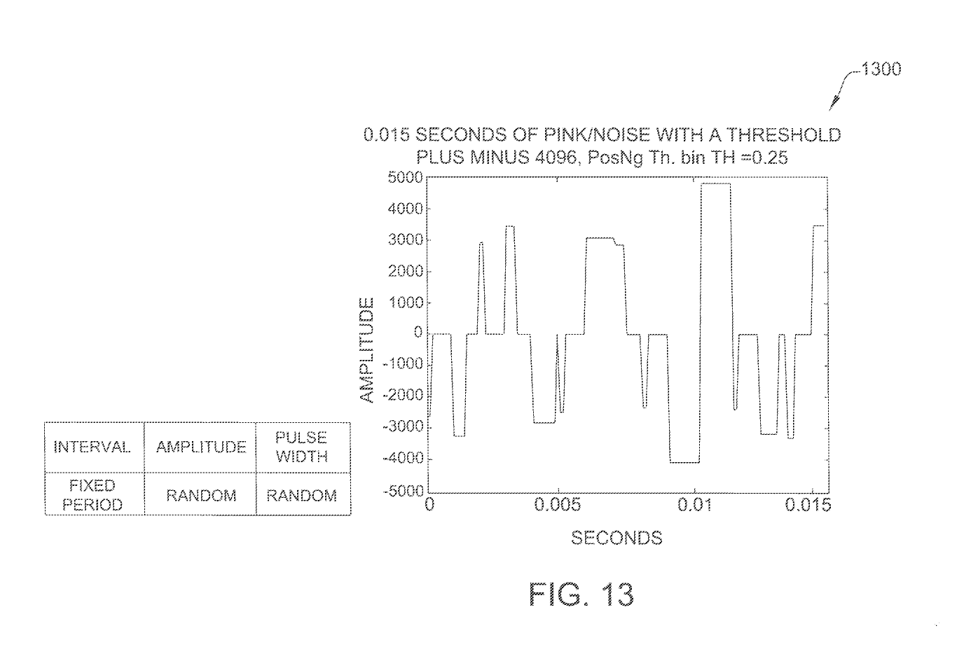

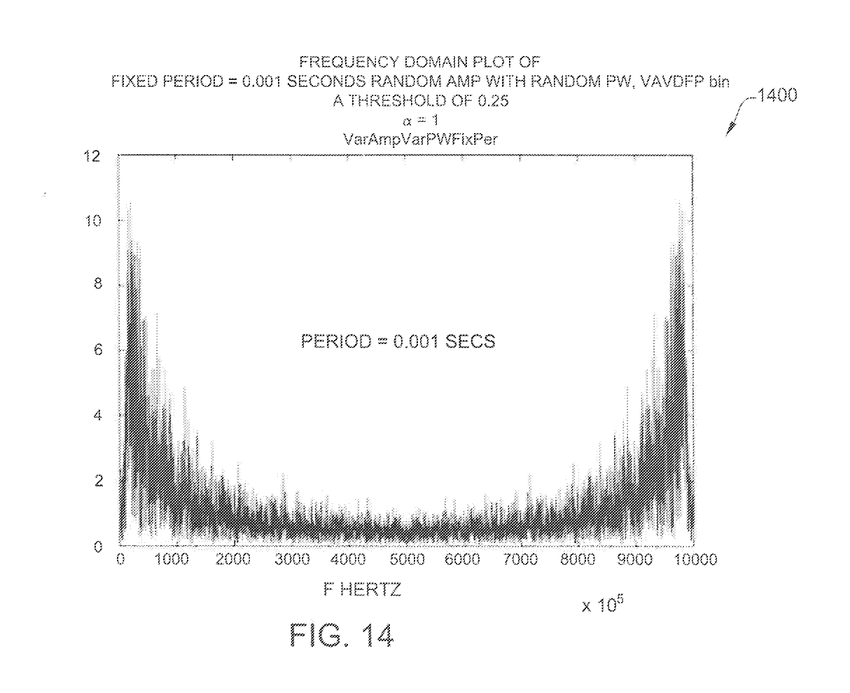

FIG. 13 is a graph 1300 showing a modified final pink noise signal in which the interval between pulses is set equal to a predetermined fixed period (e.g., 0.0001 seconds). As used herein, the ‘period’ refers to the time between the beginning of an initial pulse and the beginning of a subsequent pulse. FIG. 14 is a graph 1400 showing the frequency content of the modified final pink noise signal of graph 1300. The frequency content in graph 1400 maintains the 1/f characteristics.

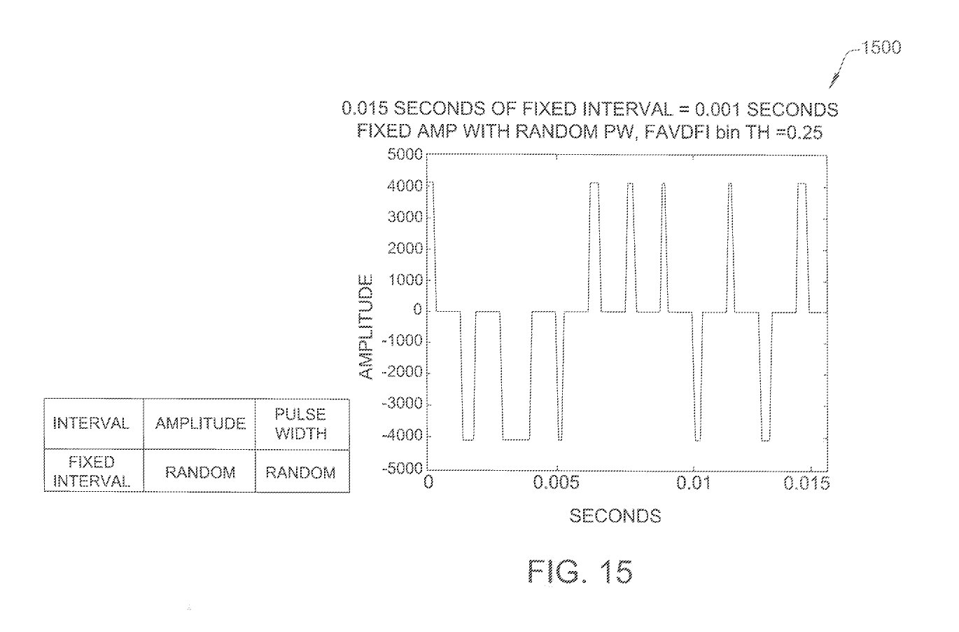

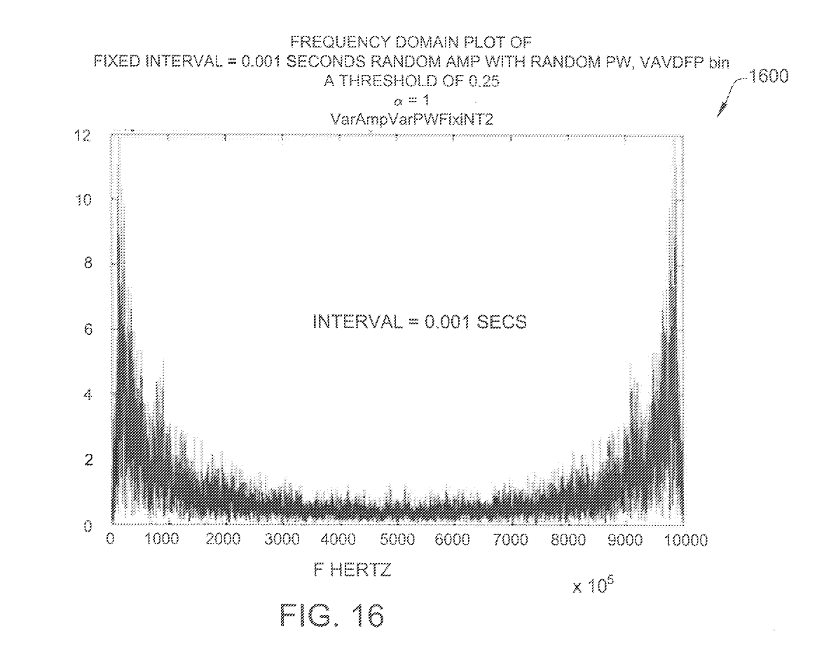

FIG. 15 is a graph 1500 showing a modified final pink noise signal in which the interval between pulses is set equal to a predetermined fixed interval (e.g., 0.0001 seconds). As used herein, the ‘interval refers to the time between the end of an initial pulse and the beginning of a subsequent pulse. FIG. 16 is a graph 1600 showing the frequency content of the modified final pink noise signal of graph 1500. The frequency content in graph 1600 maintains the 1/f characteristics.

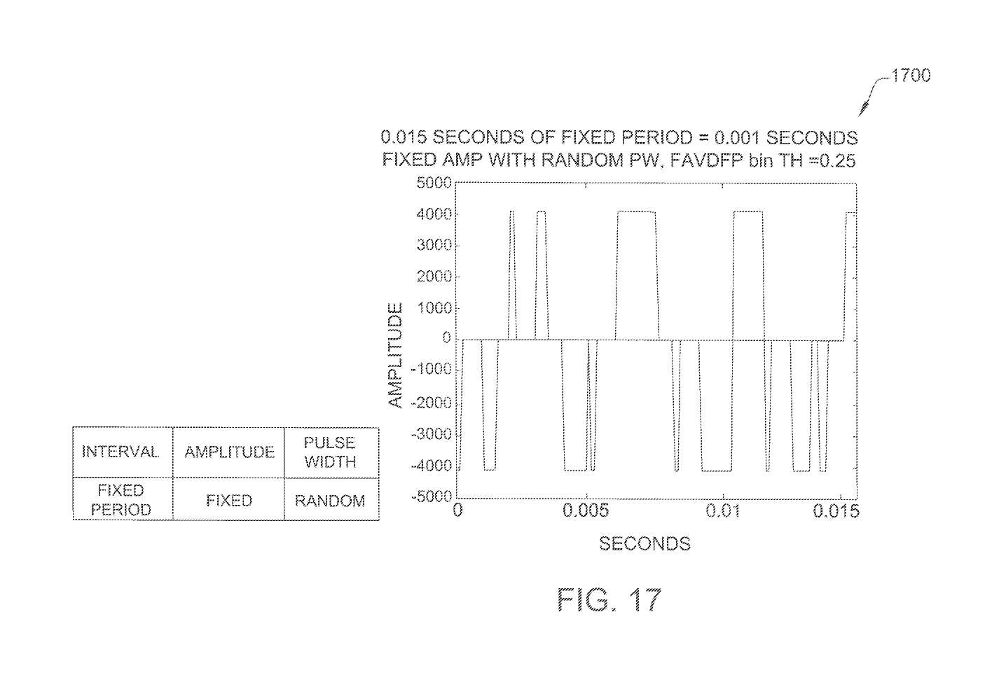

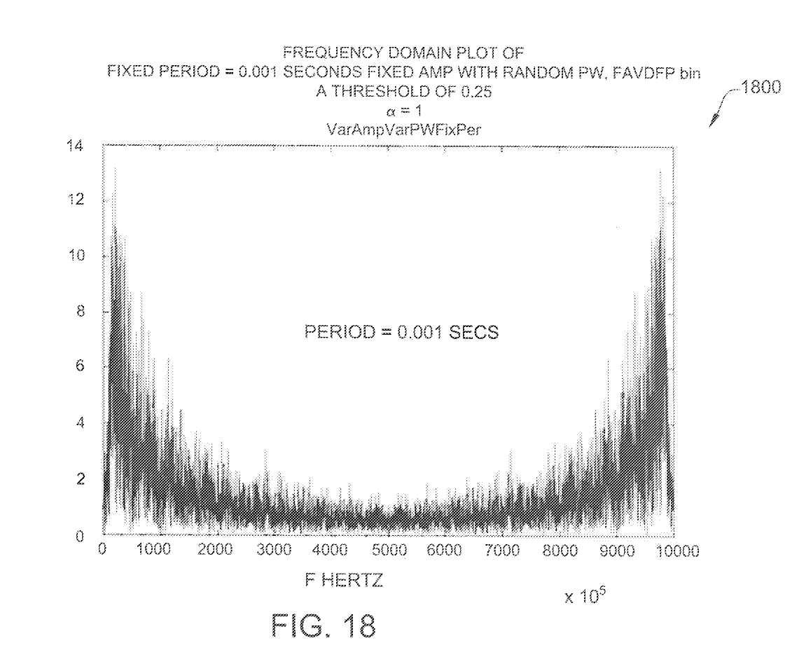

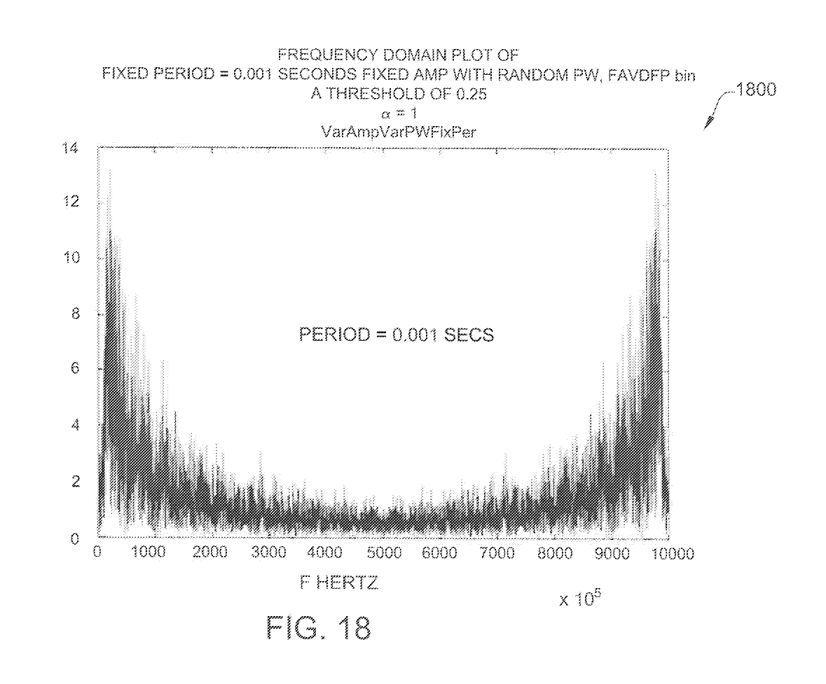

FIG. 17 is a graph 1700 showing a modified final pink noise signal in which the interval between pulses is set equal to a predetermined fixed period and the amplitude of each pulse is set equal to a predetermined value. FIG. 18 is a graph 1800 showing the frequency content of the modified final pink noise signal of graph 1700. The frequency content in graph 1800 maintains the 1/f characteristics.

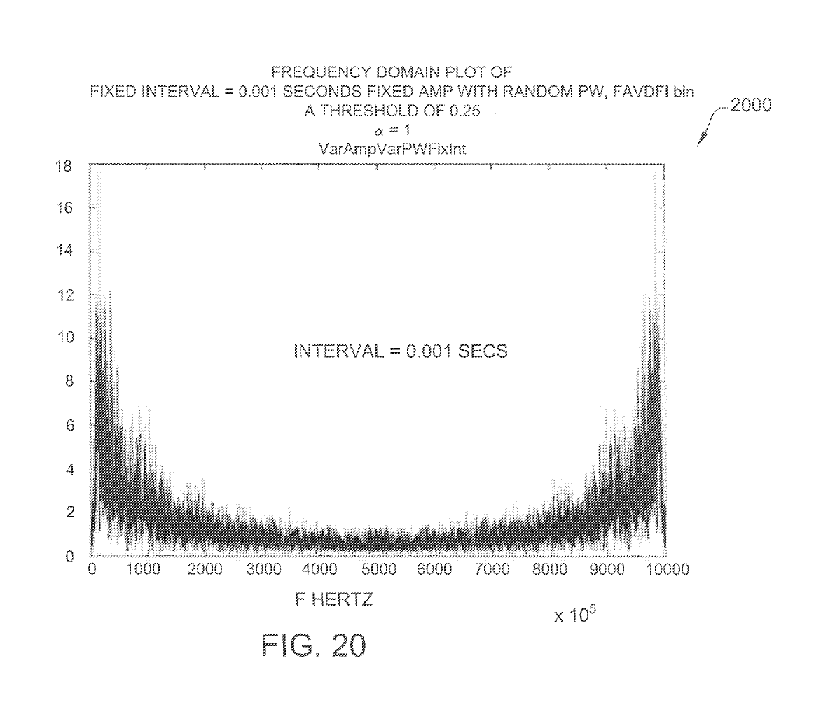

FIG. 19 is a graph 1900 showing a modified final pink noise signal in which the interval between pulses is set equal to a predetermined fixed interval and the amplitude of each pulse is set equal to a predetermined value. FIG. 20 is a graph 2000 showing the frequency content of the modified final pink noise signal of graph 1900. The frequency content in graph 2000 maintains the 1/f characteristics.

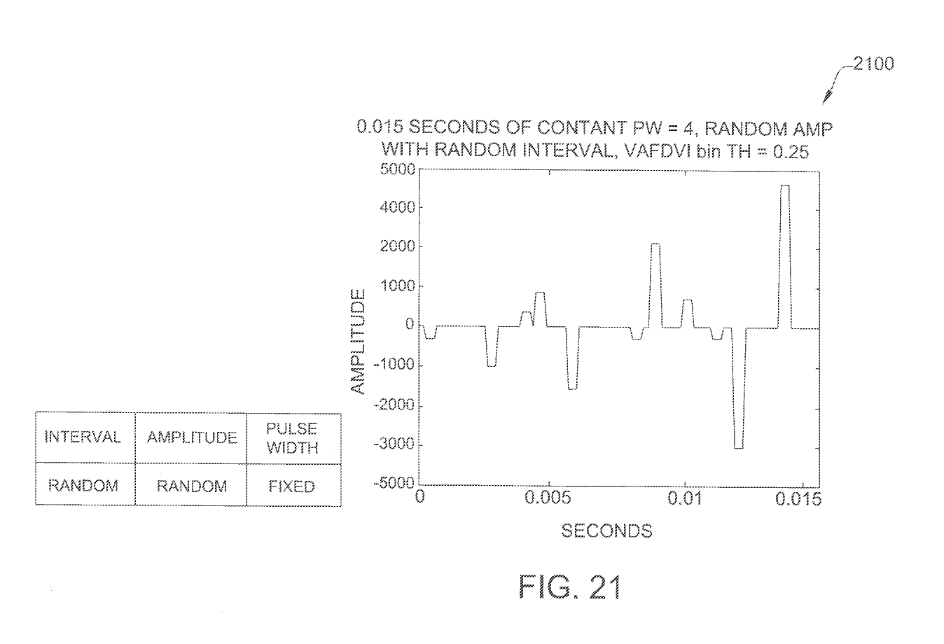

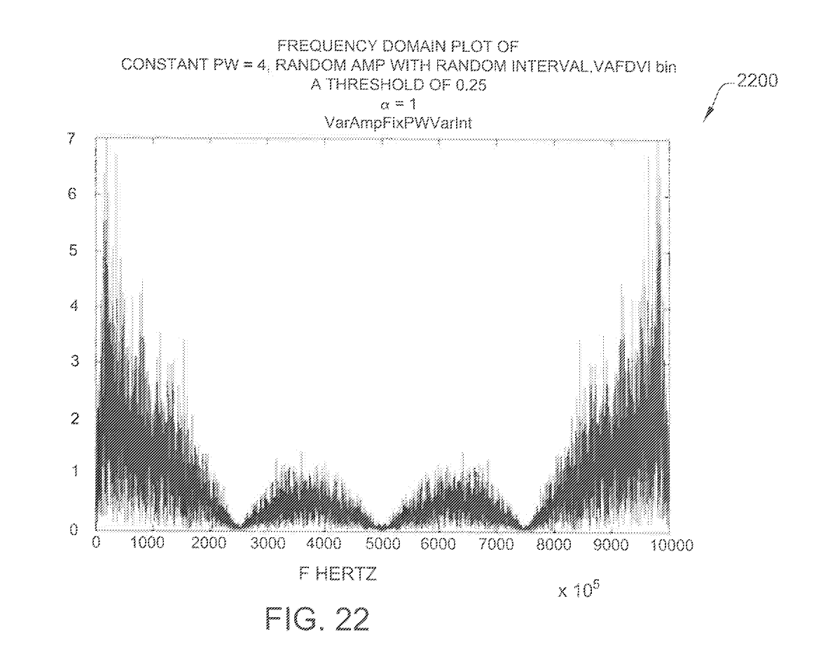

FIG. 21 is a graph 2100 showing a modified final pink noise signal in which the pulse width of each pulse is set equal to a predetermined width (e.g., 4). FIG. 22 is a graph 2200 showing the frequency content of the modified final pink noise signal of graph 2100. Notably, the frequency content in graph 2200 does not maintain the 1/f characteristics. Instead, the frequency content includes multiple zero points and lobes that are dependent on the pulse width, and does not diminish with 1/f. Accordingly, the final pink noise signal in graph 2100 is a relatively poor approximation of true pink noise. Even if the intervals between pulses and/or pulse amplitudes are controlled in combination with the pulse width, the final pink noise signal remains a relatively poor approximation of true pink noise.

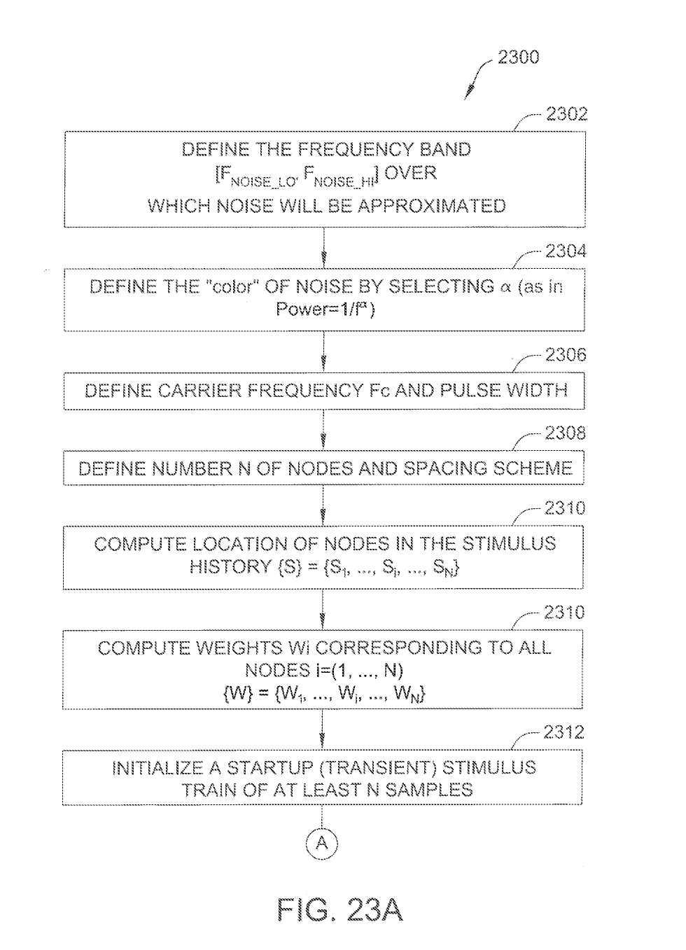

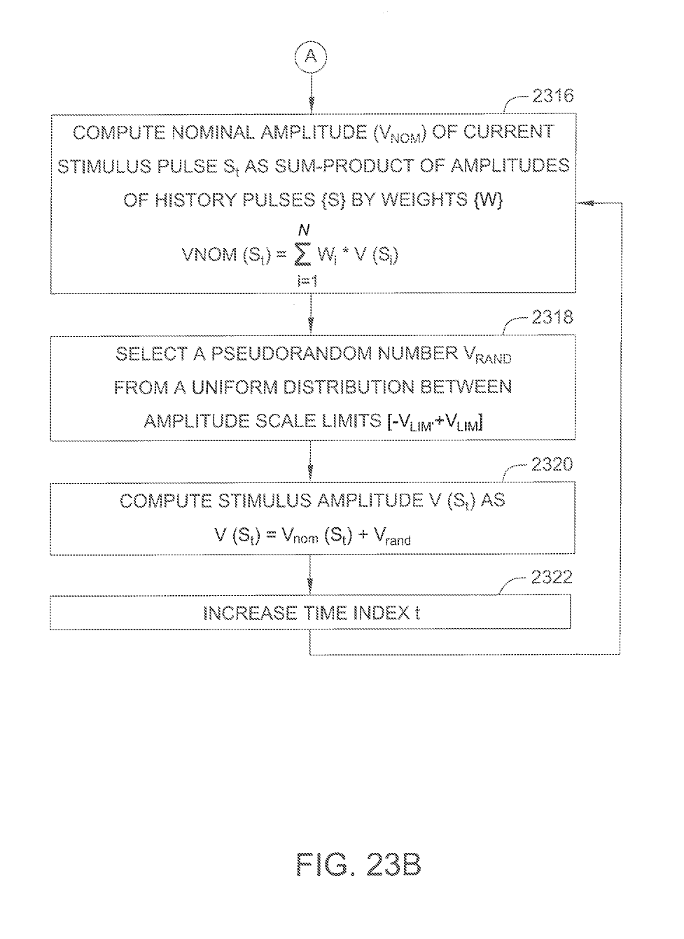

FIGS. 23A and 23B are a flow chart of another embodiment of a method 2300 for generating a pink noise signal. Method 2300 may be performed, for example, using computing device 200 (shown in FIG. 2). In general, method 2300 approximates the “memory” of noise by taking a value one time period back (for any given frequency) and adds a relatively small random number to that value. This is done for all frequencies within a selected frequency band, and the partial values are summed.

At block 2302, a frequency band is defined over which the pink noise signal will be generated. The frequency band ranges from a minimum frequency (i.e., Fₙₒᵢₛₑ_{_}ₗₒ) to a maximum frequency (i.e., Fₙₒᵢₛₑ_{_}ₕᵢ). The frequency band may be, for example, 0.1 Hz-100 Hz. At block 2304, the “color” of the noise is defined by selecting α (i.e., in 1/f^{α}).

At block 2306, a carrier frequency and pulse width are selected. The carrier frequency may be, for example, at least twice the maximum frequency of the frequency band. In general, a pulse width resulting in less than a 50% duty cycle is sufficient. Further, for improved energy utilization on the stimulation device, pulse width may be made as short as possible (e.g., at or below chronaxie).

At block 2308, a number of nodes is defined and a spacing scheme for the nodes is defined. The number of nodes may be, for example, thirty nodes. The number of nodes may be pre-defined and/or user-selectable. Nodes correspond to frequencies within the noise approximation. In one example, the frequency value at each node is “rounded” such that the value corresponds to a % number of periods of the carrier frequency and is then interpolated to the nearest frequency. Further, in one example, the node spacing may be logarithmic such that nodes are closer together at lower frequencies. This is advantageous for α>0. At block 2310, the location of each node is computed. In other embodiments, the node spacing may be uniform, or any other suitable spacing.

At block 2312, a weight is computed for each node. The weight at each node should fit the 1/f^{α} curve. At block 2314, a startup signal is initiated. The startup signal may be, for example, a zero amplitude signal initially.

Over time, a pink noise signal is generated using an iterative algorithm. Specifically, at block 2316, a nominal amplitude of each current stimulus pulse, Sₜ, is computed as the sum-product of the amplitudes of pulses at nodes in the past by the node weights computed at block 2312. At block 2318, a pseudorandom number, V_{rand}, is selected from a uniform distribution between upper and lower limits +Vₗᵢₘ and −Vₗᵢₘ. At block 2320, the stimulus amplitude is computed as the sum of the nominal amplitude from block 2316 and the pseudorandom number from block 2318. Finally, at block 2322, time is incremented and flow returns to block 2316.

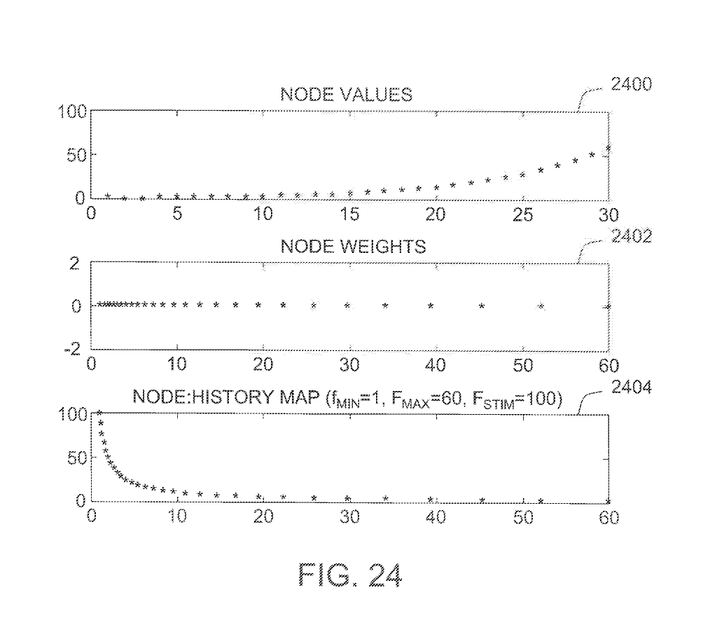

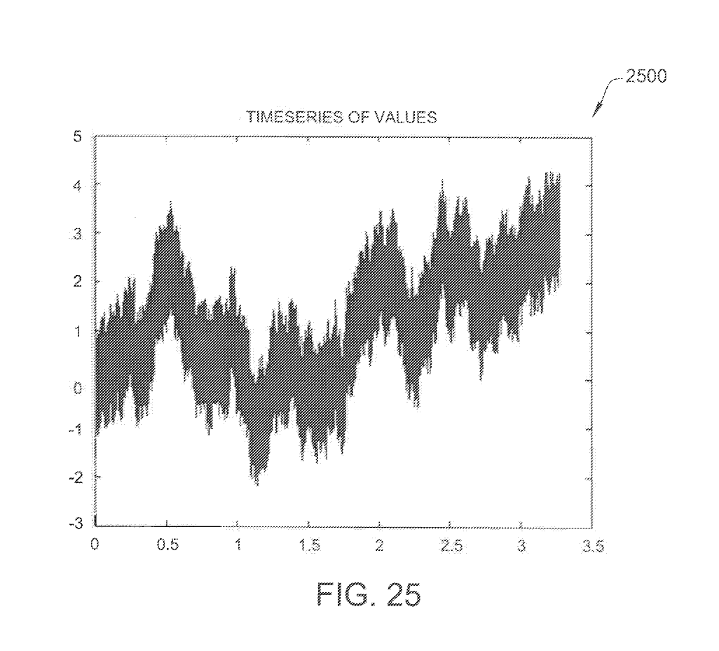

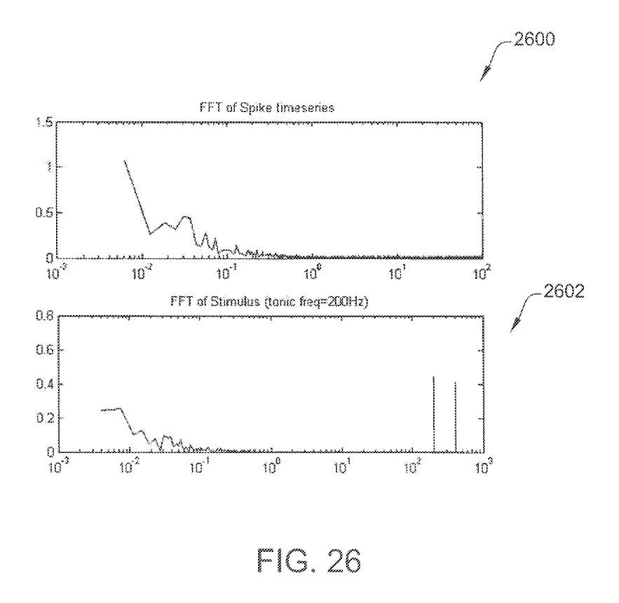

Over time, method 2300 generates a pink noise signal that may be used to control stimulation. FIG. 24 includes a first graph 2400 showing node locations, a second graph 2402 showing node weights, and a third graph 2404 showing a node history map according to one example. In this example, the number of nodes is thirty, and the nodes are spaced logarithmically. Alternatively, any number of nodes may be used and any suitable spacing may be used. FIG. 25 is a graph 2500 showing the timer-series amplitudes V(Sₜ) according to the example. FIG. 26 includes a first graph 2600 showing the FFT of the time-series of amplitudes shown in graph 2500, and a second graph 2602 showing the FFT of the pulse train generated based on those amplitudes. As demonstrated by graphs 2600 and 2602, the stimulus trains approximate pink noise. Further, as the carrier frequency and number of nodes are increased, the stimulus trains approximate pink noise even better.

The embodiments described herein provide systems and methods for generating a pink noise signal for use in a neurostimulation system. The pink noise signal includes a plurality of pulses that approximate true pink noise. The pink noise signal may be generated, for example, using an internal pulse generator or an external pulse generator.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A neurostimulation system for delivering pink noise stimulation, the neurostimulation system comprising:
a stimulation lead comprising a plurality of contacts; and
a pulse generator communicatively coupled to the stimulation lead and configured to:
generate white noise;
filter the generated white noise to generate pink noise;
apply a high pass filter to the pink noise;
apply thresholding to the filtered pink noise;
convert each excursion in the thresholded pink noise into a square pulse to generate a final pink noise signal; and
cause stimulation to be delivered by the stimulation lead based on the final pink noise signal.

2. The neurostimulation system of claim 1, wherein to convert each excursion into a square pulse, the pulse generator is configured to convert each excursion into a square pulse such that each pulse has a predetermined amplitude.

3. The neurostimulation system of claim 1, wherein to convert each excursion into a square pulse, the pulse generator is configured to convert each excursion into a square pulse such that there is a predetermined fixed period between subsequent pulses.

4. The neurostimulation system of claim 1, wherein to convert each excursion into a square pulse, the pulse generator is configured to convert each excursion into a square pulse such that there is a predetermined fixed interval between subsequent pulses.

5. The neurostimulation system of claim 1, wherein the pulse generator is an internal pulse generator.

6. The neurostimulation system of claim 1, wherein the pulse generator is an external pulse generator.

7. The neurostimulation system of claim 1, wherein to filter the white noise, the pulse generator is configured to filter the white noise using a 1/f^{α} filter, where a is a predetermined constant.

8. A computer-implemented method for generating a pink noise signal, the method comprising:
generating, using a computing device, white noise using a random number generator;
filtering, using the computing device, the generated white noise to generate pink noise;
applying, using the computing device, a high pass filter to the pink noise;
applying, using the computing device, thresholding to the filtered pink noise; and
converting, using the computing device, each excursion in the thresholded pink noise into a square pulse to generate a final pink noise signal.

9. The method of claim 8, further comprising delivering, using a neurostimulation system, stimulation based on the final pink noise signal.

10. The method of claim 8, wherein converting each excursion into a square pulse comprises converting each excursion into a square pulse such that each pulse has a predetermined amplitude.

11. The method of claim 8, wherein converting each excursion into a square pulse comprises converting each excursion into a square pulse such that there is a predetermined fixed period between subsequent pulses.

12. The method of claim 8, wherein converting each excursion into a square pulse comprises converting each excursion into a square pulse such that there is a predetermined fixed interval between subsequent pulses.

13. The method of claim 8, wherein the computing device is a pulse generator for use in a neurostimulation system.

14. The method of claim 8, wherein filtering the white noise comprises filtering the white noise using a 1/f^{α} filter, where α is a predetermined constant.

15. A computer-implemented method for generating a pink noise signal, the method comprising:
determining, using a computing device, a frequency band;
determining, using the computing device, a number of nodes and a spacing scheme for the nodes;
computing, using the computing device, a location of each node within the frequency band;
computing, using the computing device, a weight for each node;
initiating, using the computing device, a startup signal;
computing, after a predetermined period of time, a nominal amplitude of a current stimulus pulse as a sum-product of amplitudes of previous pulses by the computed weights;
selecting a pseudorandom number; and
computing a final stimulus amplitude as a sum of the nominal amplitude and the pseudorandom number to generate the pink noise signal.

16. The method of claim 15, further comprising delivering, using a neurostimulation system, stimulation based on the pink noise signal.

17. The method of claim 15, wherein determining a spacing scheme for the nodes comprises determining a logarithmic spacing scheme.

18. The method of claim 15, wherein determining a spacing scheme for the nodes comprises determining a uniform spacing scheme.

19. The method of claim 15, wherein the computing device is a pulse generator for use in a neurostimulation system.

20. The method of claim 15, wherein initializing a startup signal comprises initiating a zero amplitude signal.
